(19) United States
(12) Patent Application Publication
(10) Pub. No.: US 2010/0132253 A1
(43) Pub. Date: Jun. 3, 2010

(54) FUEL ADDITIVES AND FUEL COMPOSITIONS AND METHODS FOR MAKING AND USING THE SAME

(75) Inventors: Benjamin J. KAUFMAN, Hopewell Junction, NY (US); James R. KETCHAM, Salt Point, NY (US); William P. ACKER, Rexford, NY (US)

Correspondence Address: HESLIN ROTHENBERG FARLEY & MESITI PC, 5 COLUMBIA CIRCLE, ALBANY, NY, 12203, US

(73) Assignee: TACONIC ENERGY, INC., Salt Point, NY (US)

(21) Appl. No.: 12/327,135

(22) Filed: Dec. 3, 2008

(51) Int. Cl. C10L 1/22; C07C 233/01

(52) U.S. Cl. 44/418; 564/224

(57) ABSTRACT

The present invention provides friction-modifying fuel additives and fuel compositions including an alkanolamine, which are effective for enhancing engine performance wherein the additive is free of esters or the ratio of amides to esters is greater than 1.4 to 1 and methods for making and using the same.

### BACKGROUND OF THE INVENTION

1. Field of the Invention

The present invention relates to fuel additive compositions and methods for making and using same. More particularly, the present invention relates to friction-modifying fuel additives and fuel compositions containing such additives. The additive comprises alkanolamides therein and is either free of esters or has an amide to ester ratio of greater than 1.4 to 1, that improve engine performance as well as provide fuel economy benefits.

2. Description of the Related Art

Fuel economy standards mandated by the federal government have resulted in efforts by the automotive industry to improve the fuel economy of motor vehicles. One way to reduce fuel consumption is by reducing friction in particular areas of the engine, e.g., bearings, valve trains, pistons, rings, and oil pumps. A decrease in the friction of these components will be reflected in a corresponding fuel economy improvement. Therefore, there has been an ongoing search for friction modifier compositions that will decrease friction in these key areas and thus improve fuel economy. Various types of additives have been used as friction modifiers. Some of the more commercially and conventionally used friction modifiers are fatty acid esters, fatty acid amides and fatty acid ester-amides.

As discussed at some length in U.S. Pat. No. 6,277,158 to McLean, the performance of gasolines and other fuels can be improved through the use of additive technology. For instance, detergents have been used to inhibit the formation of intake system deposits (IVD), and thereby improve engine cleanliness and performance. Regulatory mandates have required the introduction of low sulfur fuels, which are known to be less lubricating and raise concerns regarding the durability of fuel pumps and injectors. Sulfur itself is not directly known to be a lubricity modifying agent. However, removal of sulfur by deep hydrotreating is known to also inadvertently remove natural lubricity components of the fuel, such as certain aromatics, carboxylic acids, and esters. Unfortunately, commercial gasoline detergents and dispersants generally show very little friction reducing characteristics until very high concentrations of them are added to the fuel. These high detergent concentrations often reach levels where no-harm effects such as combustion chamber deposits (CCD) become unacceptable.

It has been suggested that separate friction modifiers can be added to gasoline to increase fuel economy by reducing engine friction. Fuel friction modifiers would also serve to protect high-pressure fuel pumps and injectors such as those found in direct injection gasoline (DIG) engines from wear caused by fuel. Worldwide regulations calling for a steep reduction in fuel sulfur levels may exacerbate this wear problem even further. In selecting suitable components for a combined detergent/friction modifier additive package it is important to ensure a balance of detergent and friction modification properties, and so forth. Ideally, the friction modifier should not adversely affect the deposit control function of the detergent. In addition, the additive package should not adversely effect on engine performance. For example, the additive package should not promote valve sticking or cause other performance-reducing problems. To be suitable for commercial use, the friction modifier additive also must pass all no-harm testing required for gasoline performance additives. This is often the biggest hurdle for commercial acceptance. The no-harm testing involves 1) compatibility with gasoline and other additives likely to be in gasoline at a range of temperatures, 2) no increase in IVD and CCD, 3) no valve stick at low temperatures, 4) no corrosion in the fuel system, cylinders, and crankcase, and 5) no adverse reactions during storage or handling in distribution systems. Developing an additive meeting all these criteria is challenging.

A number of prior friction modifiers for fuels have been natural product derived (plant and animal sourced) fatty esters or oils, with only a few purely synthetic products. For example, WO 01/72930 A2 describes a mechanistic proposal for delivery of a fuel born friction modifier to the upper cylinder wall and into the oil sump resulting in upper cylinder/rings and valves lubrication. The friction modifier is packaged with fuel detergent dispersants such as polyetheramines (PEAs), polyisobutene amines (PIBAs), Mannich bases, and succinimides. Fuel friction modifier prior art identified in the WO '930 reference include U.S. Pat. Nos. 2,252,889, 4,185,594, 4,208,190, 4,204,481, and 4,428,182, which all describe use of fuel modifiers in diesel fuel. Chemistries covered by these patents include fatty acid esters, unsaturated dimerized fatty acids, primary aliphatic amines, fatty acid amides of diethanolamine and long-chain aliphatic monocarboxylic acids. Another specific mentioned patent therein is U.S. Pat. No. 4,427,562, which discloses a lubricant oil and fuel friction modifier made by reacting primary alkoxyalkylamines with carboxylic acids or by aminolysis of the appropriate formate ester.

U.S. Pat. No. 4,729,769, and related U.S. Pat. Nos. 6,743,266 and 6,835,217, describes a gasoline carburetor detergent for gasoline compositions derived from reaction products of a C₆ -C₂₀ fatty acid ester, such as coconut oil, and a mono- or di-hydroxy hydrocarbyl amine, such as diethanolamine, as carburetor detergents. The additive in the '769 patent is described as being useful in any gasoline including leaded and those containing methylcyclopentadienyl manganese tricarbonyl (MMT). The fuel described in the '769 patent may contain other necessary additives such as anti-icers, and corrosion inhibitors.

U.S. Patent Application Publication US 2008/0072477 discloses alkanolamide and their use as fuel additives formed by the reaction product of a fatty acid and diethanolamine (DEA).

U.S. Pat. No. 5,858,029 describes friction reducing additives for fuels and lubricants involving the reaction products of primary etheramines with hydrocarboxylic acids to give hydroxyamides that exhibit friction reduction in fuels and lubricants. Other prior patents describing friction modifiers include U.S. Pat. No. 4,617,026 (monocarboxylic acid of ester of a trihydric alcohol, glycerol monooleate as fuels and lubricant friction modifier); U.S. Pat. Nos. 4,789,493, 4,808,196, and 4,867,752 (use of fatty acid formamides); U.S. Pat. No. 4,280,916 (use of fatty acid amides); U.S. Pat No. 4,406,803 (use of alkane 1,2-diols in lubricants to improve fuel economy); and U.S. Pat. No. 4,512,903 (use of amides from mono- or polyhydroxy substituted aliphatic monocarboxylic acids and amines). U.S. Pat. No. 6,328,771 discloses fuel compositions containing lubricity enhancing salt compositions made by the reaction of certain carboxylic acids with a component that is comprised of a heterocyclic aromatic amine. EP 0 798 364 discloses diesel fuel additives comprising a salt of a carboxylic acid and an aliphatic amine, or an amide obtained by dehydration-condensation between a carboxylic acid and an aliphatic amine.

EP 0 869 163 A1 describes a method for reducing engine friction by use of ethoxylated amines. In addition, U.S. Pat. No. 4,086,172 (oil soluble hydroxyamines such as “ETHOMEEN 18-12®” formula C₁₈H₃₇ N—(CH₂CH₂ OH)₂ as lubricant antioxidant); U.S. Pat. No. 4,129,508 (reaction products of succinic acid or anhydride and a polyalkylene glycol or monoether, an organic basic metal, and an alkoxylated amine as a demulsifier); U.S. Pat. Nos. 4,231,883; 4,409,000; and 4,836,829, all teach various uses of hydroxyamines in fuels and lubricants.

U.S. Pat. No. 6,277,158 describes the current practice in the supply of gasoline as generally being to pre-mix the fuel additives into a concentrate in a hydrocarbon solvent base, and then to inject the concentrate into gasoline pipelines used to fill tankers prior to delivery to the customer. To facilitate injection of the concentrate into the gasoline, it is important that the concentrate be in the form of a low viscosity, homogeneous liquid.

Further, U.S. Pat. No. 4,960,530 discloses an example of a lubricant additive and composition that can reduce friction. Hydroxyalkyl amides have been used extensively as friction modifiers. U.S. Pat. No. 4,208,293 discloses a lubricating oil adapted for use as a crankcase lubricant in internal combustion engines containing a friction-reducing amount of a fatty acid amide or ester of diethanolamine.

Despite the wide variety of additives available for lubricant and fuel applications, there remains a need for more effective additives which can provide enhanced fuel economy, and improved engine performance.

### SUMMARY OF THE INVENTION

In one aspect, the application for patent provides a fuel additive for use in fuel compositions. In one embodiment, for example, the fuel additive includes alkanolamides therein, wherein the fuel additive is free of esters or, if esters are present therein the molar ratio of amides to esters in the additive is greater than 1.4 to 1. The alkanolamides may be produced from alkanolamines including diethanolamine (DEA).

In another aspect, the invention provides a fuel composition for an internal combustion engine having a major amount of a hydrocarbon fuel and a minor amount of fuel additive that includes alkanolamides therein, wherein the fuel additive is free of esters or, if esters are present therein, the molar ratio of amides to esters in the additive is greater than 1.4 to 1.

In yet another aspect, the invention provides methods for making a fuel additive for use in a fuel composition by reacting a fatty acid, fatty acid ester or natural or synthetic oil, with an alkanolamine. In this embodiment, the fatty acid, fatty acid ester or natural or synthetic oil is reacted with alkanolamine to produce a reaction product containing alkanolamides. The fatty acid, fatty acid ester, and/or oil may be mixed with an amount of a gasoline compatible solvent, with or without the presence of a hydrophobic acid catalyst. The reaction is carried out at elevated temperatures until the desired alkanolamides are formed and water, methanol or glycerin is removed from the system. Esters including ester amines may also be formed from the reaction. Such esters may either be removed from the additive, and/or if present in the additive, the molar ratio of amides to esters is preferably greater than 1.4 to 1.

In another aspect, the invention provides methods for enhancing fuel economy of an internal combustion engine by combusting in the engine a hydrocarbon fuel comprising a major amount of a hydrocarbon fuel, and a minor amount of a fuel additive as described herein. In one embodiment, the hydrocarbon fuel can include gasoline, diesel fuel, kerosene or jet fuel. The fuel and/or additive includes alkanolamides therein may be free of esters, or if esters are present, in the molar ratio of amides to esters in the additive is greater than 1.4 to 1.

In the embodiments of the invention, the fuel economy benefits improve as a function of the increase in amide to ester molar ratio. Generally, the higher the amide to ester molar ratio, the better the fuel economy. Therefore, it is preferred to have the additive be free of esters, or if the esters are present, the molar ratio of amides to esters should be greater than 1.4 to 1, preferably, greater than 2 to 1, more preferably greater than 4 to 1, more preferably greater than 9 to 1 (or between 9 to 1 and 20 to 1), or even more preferably greater than 50 to 1 (or between 50 to 1 and 99 to 1); or even more preferably greater than 99 to 1.

These and other aspects of the invention will become evident upon reference to the following detailed description of the invention and accompanying Examples.

### BRIEF DESCRIPTION OF THE DRAWINGS

FIG. 1 depicts graphical results for a HWFET test of the fuel additive at 1000 ppm concentration.

FIG. 2 depicts graphical results for a HWFET test for the fuel additive at 500 ppm.

### DETAILED DESCRIPTION OF THE INVENTION

The present invention provides fuel additive compositions and methods for making and using same. More specifically, the present invention provides friction-modifying additives for fuel compositions having alkanolamides therein, and is either ester free, or if esters are present, has an amide to ester molar ratio of greater than 1.4 to 1 which is effective for lowering friction and enhancing fuel economy and engine performance.

### Friction Modifier

In one aspect, the application for patent provides a fuel additive for use in fuel compositions. The present fuel additive or fuel composition has a molar ratio of amides to esters of greater than 1.4 to 1. As used herein, the expression amide is used in its broadest sense to include compounds generally represented by the formula: R₁(CO)NR₂R₃, where either or both R₂ and R₃ may be or include hydrogen. Specifically, an amide can also be regarded as a derivative of a carboxylic acid in which the hydroxyl group has been replaced by a nitrogen containing material the simplest form being ammonia. Amides, including alkanolamides, are commonly formed from the reaction of a carboxylic acid group with an amine.

Generally, as used herein, the expression alkanolamides is used in its broadest sense to refer to a class of compounds which contain both an amide and hydroxyl functionality. Alkanolamides are generally produced by the reaction of a fatty acid, methyl ester or oil with an alkanolamine. Alkanolamines carry hydroxyl (—OH) and amino (—NH₂, —NHR, and —NR₂) functional groups on an alkane backbone. They are sometimes referred to as fatty alkanolamides.

The fuel additive may be obtained from at least one product of a reaction of a natural or synthetic oils, fatty acids (such as carboxylic acid) and esters (such as a methyl ester), with an alkanolamine.

Alkanolamides may be prepared commercially from alkanolamines, such as diethanolamine (DEA), by reaction at elevated temperatures. Reacting a fatty acid, oil, and/or ester with diethanolamine may produce numerous side reactions. The reaction at temperatures typically over 120° C. of approximately equimolar amounts of an alkanolamine and carboxylic acid derived material result in superamides products. These superamides products typically contain both amide and ester products. By contrast, using excess alkanolamine, ester starting material, catalysts and/or controlled reaction conditions, results in a predominantly amide product with very little of the corresponding ester may result. These amide products are generally referred to Kritchevsky amides.

In accordance with the principles of the present invention, a fuel additive having a high amide to ester ratio may be produced via the aforementioned reaction. For example, by reacting a high molar amount of amine with an acid of lower molar amount, a reaction product comprising fatty acid alkanolamides and ester and amide mixtures, fatty acid salts, and diethanolamine may be produced. The reaction occurs in accordance with the following equation:

RCO₂H+HN(CH₂CH₂OH)₂→RCON(CH₂CH₂OH)₂+H₂O+RCO₂H+HN(CH₂CH₂OH)₂+RCO₂CH₂CH₂NHCH₂CH₂OH+RCO₂CH₂CH₂NHCH₂CH₂O(C═O)R

For example, when using two moles of amine and reacting the same with one mole of acid, the reaction produces approximately 60 to 65% alkanolamide. Such a reaction may be performed at 150° to 200° C. However, it may be desirable to perform the reaction at less than 150 degrees C. to minimize certain known byproducts.

The aforementioned reaction may also be performed using the methyl ester of the fatty acid as a starting material and reacting to the same with an alkanolamine. By using a basic catalyst such as sodium methoxide or CaO or ZnCl₂ or a purified fatty acid, alkanolamide may be produced at reaction temperatures of less than 150° C., for example at a 100° C., and the methanol distilled during the reaction. Such a reaction occurs according to the following formula:

RCO₂CH₃+HN(CH₂CH₂OH)₂→RCON(CH₂CH₂OH)₂+CH₃OH

Such high purity fatty acid alkanolamides may be prepared from the methyl esters of various acids including lauric, myristic, palmitic, steric, oleic, linoleic, erucic, and dimeric acids. In addition, natural or synthetic oils may also be used as the reaction starting material. In this situation, glycerin is created as a by-product, which may be removed therefrom.

The reactants may include organic acid compounds containing at least one carboxyl group (—C(═O)OH). In some embodiments, the carboxylic acid is chosen from a C₆-C₂₂ carboxylic acid. In other embodiments, the carboxylic acid is chosen from C₁₂-C₁₈ carboxylic acids. For example, in a particular embodiment, the carboxylic acid can include lauric acid (CH₃(CH₂)₁₀COOH), while in another embodiment the carboxylic acid includes myristic acid (CH₃(CH₂)₁₂COOH) and stearic acid (CH₃(CH₂)₁₆COOH) or combinations thereof.

The reactants may be derived from the group consisting of canola oil, castor oil, cocoa butter, coconut oil, cotton seed oil, olive oil, palm kernel oil, palm kernel (olein), palm kernel (stearine), peanut oil, rapeseed oil, safflower oil, soybean oil, sunflower oil, tall oil, corn oil, butter, lard, tallow, yellow grease, blubber, herring, menhaden, sardine oil, babasso oil and mixtures thereof and may be selected from the group consisting of lauric, cis-9-dodecenoic, myristic, myristoleic, cis-9-tetradecenoic, pentadecanoic, palmitic, palmitoleic, cis-9-hexadecenoic, heptadecanoic, heptadecenoic, steric, oleic, linoleic, linolenic, ricinoleic, dihydroxystearic, and mixtures thereof.

The alkanolamine which is reacted with the fatty acid, oil or ester may be a primary or secondary amine which possesses at least one hydroxyl group. The alkanolamine corresponds to the general formula HN(R′OH)₂₋ₓHₓ wherein R′ is a lower hydrocarbyl having from about two to about six carbon atoms and x is 0 or 1. The expression “alkanolamine” is used in its broadest sense to include compounds containing at least one primary or secondary amine and at least one hydroxyl group such as, for example, monoalkanolamines, dialkanolamines, and so forth. It is believed that almost any alkanolamine can be used, although preferred alkanolamines are lower alkanolamines having from about two to about six carbon atoms. The alkanolamine can possess an O or N functionality in addition to the one amino group (that group being a primary or secondary amino group) and the at least one hydroxyl group. Suitable alkanolamines for use herein include monoethanolamine, diethanolamine (DEA), propanolamine, isopropanolamine, dipropanolamine, di-isopropanolamine, butanolamines, aminoethylaminoethanols, e.g., 2-(2-aminoethylamino)ethanol, and the like with diethanolamine being preferred. It is also contemplated that mixtures of two or more alkanolamines can be employed.

In general, the reaction can be conducted by heating the mixture of oil, fatty acid or ester in the desired ratio to produce the reaction products wherein the molar ratio of amides to esters is greater than 1.4 to 1, 2 to 1, 4 to 1, 9 to 1, 20 to 1, 30 to 1, 50 to 1, and greater than 99 to 1. The reaction can be carried out with or without a hydrophobic acid catalyst and is monitored to assure that the starting materials are substantially transformed into the desired amide esters and ester amines. This conversion can typically be conducted by maintaining the reactants at a temperature of from about 80° C.-200° C. and preferably from about 140° C.-160° C. for a time period ranging from about 1-10 hours and preferably from about 2-6 hours. Such catalysts may include aromatic sulfonic acid such as but not limited to benzene or naphthalene sulfonic acid and aliphatic sulfonic acids such as but not limited to dodecylsulfonic acid.

If desired, the reaction can be carried out in a solvent, preferably one which is compatible with the ultimate composition in which the product is to be used. Useful solvents include, but are not limited to, Aromatic-100, Aromatic-150, Shellsolv AB, Avjet, toluene, xylene, tetrahydrofuran, isopropanol isobutylcarbinol, n-butanol, and petroleum hydrocarbon solvents such as solvent naphtha, and the like and mixtures thereof.

It will be understood by those skilled in the art that the foregoing reaction will produce a complex mixture of compounds including fatty acid amides, fatty acid esters, fatty acid ester-amides, and unreacted starting reactants.

For example, reaction includes alkanolamides, as follows:

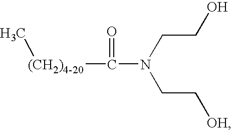

and esters, as follows:

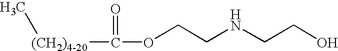

and di-esters, as follows:

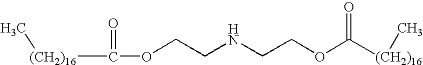

In accordance with the principles of the present invention, these reaction products may be useable as a fuel additive, which yields effective and unexpected improved friction modification when the molar ratio of amides, including but not limited to alkanolamides, to esters is maximized in such an additive, and/or the esters eliminated therefrom. The ratio of amides to esters may be increased in a variety of ways using excess or increased amounts of alkanolamine, in addition to using a catalyst with a methyl ester as a reactant. This reaction can be run at temperatures below 100 C. The reaction of a fatty acid with excess of alkanolamine can also yield the amide products with the reactions typically run at 140-160 C. In addition, catalysts and controlled reaction conditions may be used to increase the amide to ester ratio. In addition, unwanted starting materials and esters products may be removed or eliminated by techniques such as water washing or filtration with an appropriate absorbant to obtain the desired maximized amide to ester ratio.

In accordance with the principles of the present invention, the molar ratio of amides to esters within the fuel additive or fuel composition may be determined by IR spectroscopy. For example, the molar amount of ester may be represented by the area within the spectral peak curve depicting ester functional groups. In addition, the molar amount of amide may be represented by the area within the spectral peak curve depicting amide functional groups. The total area of amide functionality may then be compared with the total area of ester functionality to obtain the molar amide to ester ratio. Eliminating or minimizing the esters from the additive or fuel composition, so as to increase the amide to ester ratio present therein, may achieve increased friction modification, and thus fuel economy.

The treat level of the friction modifier in the finished fuel generally will be an amount providing the improved performance effects, such as in terms of improving fuel efficiency, and so forth, as described herein. For example, a treat level of at least about 20 ppm to about 2000 ppm (relative proportion of fuel additive to hydrocarbon fuel) of the friction modifier can be used for gasolines. More specifically about 30 to about 1800 ppm, and more specifically about 40 to about 1500 ppm, and more specifically about 50 to about 1000 ppm and most preferably, a treat level of about 500 ppm is used.

### Detergent

In another embodiment, the fuel additive composition according to the present invention can include a fuel detergent. The detergent used in the fuel additive described herein may include any suitable commercially available detergent or deposit inhibitor available for this function. For example, the detergents may be present at a level of about 20-about 1000 ppm (relative proportion of the detergent to hydrocarbon fuel), as is typically well known to those skilled in the art. Deposit inhibitors for gasoline, usually referred to as detergents or dispersants, are well known and a variety of compounds can be used. Examples include, but are not limited to, any polyether amine and/or one or more of the type based on a polyolefin, e.g., polyethylene, polypropylene, polybutylene, including isomers thereof, and copolymers of at least two of the foregoing; and polyolefin-based detergents, e.g., imides such as succinimide, amines and the like where the latter may be made by chlorinating selected olefins, and reacting the thus-chlorinated olefins with polyamines, e.g., ethylenediamine, tetraethylenepentaamine, etc. A suitable selected olefin is polyisobutene having a molecular weight in the range of from 450 to 1500, and more preferably 900 to 1400. Another suitable detergent may be based on a polyisobutene, preferably of molecular weight in the range of from 450 to 1500, more preferably 900 to 1400, which has been reacted with maleic acid and the resulting acid-functionalised polyolefin thereafter reacted with a polyamine such as tetraethylenepentamine. Processes not involving chlorine are also known. For example, the OXO process used by BASF in preparing a polyolefin-amine which are commercially available as Puradd FD-100 and the like.

Another suitable detergent for use in the present invention includes Mannich bases. A preferred detergent for the additive concentrate of the present invention is a Mannich base detergent.

The Mannich base detergents suitable for use in the present invention include the reaction products of a high molecular weight alkyl-substituted hydroxyaromatic compound, aldehydes and amines. The alkyl-substituted hydroxyaromatic compound, aldehydes and amines used in making the Mannich reaction products of the present invention may be any such compounds known and applied in the art.

Suitable Mannich detergents for use in the present invention include those detergents taught in U.S. Pat. Nos. 4,231,759; 5,514,190; 5,634,951; 5,697,988; 5,725,612; and 5,876,468, the disclosures of which are incorporated herein by reference. Suitable Mannich base detergents also include, for example, HiTEC® 4995 and HiTEC® 6410 Detergents and are available from the Afton Corporation, Richmond, Va., U.S.A.

### Carrier

In certain embodiments, the detergents can be used with a carrier or induction aid. This carrier typically will be a carrier fluid. Such carriers can be of various types, such as, for example, liquid poly-α-olefin oligomers, mineral oils, liquid poly(oxyalkylene) compounds, polyalkenes, and similar liquid carriers. Mixtures of two or more such carriers can also be employed. Suitable carriers are also disclosed in U.S. Pat. No. 6,743,266, which is incorporated herein by reference.

### Optional Solvent

Among other things, the kinematic viscosity of the fuel additive can be adjusted (reduced) by solvent addition, if desired or needed. To achieve this, a solvent can be added to the reaction products, such as an aromatic hydrocarbon solvent or an alcohol. Examples of the solvents suitable for use with the present invention include, but are not limited to, one or more of Aromatic-100, Aromatic-150, Shellsolv AB, Avjet, toluene, xylene, tetrahydrofuran, isopropanol isobutylcarbinol, n-butanol, and petroleum hydrocarbon solvents such as solvent naphtha, and the like. More specifically, the molar ratio of amides to esters may be greater than 2 to 1, greater than 4 to 1, greater than 9 to 1, greater than 20 to 1, greater than 50 to 1, and greater than 99 to 1. Moreover, the molar ratio of amides to esters may be between 9 to 1 and 50 to 1, and between 50 to 1 and 99 to 1.

### Fuel Compositions

In yet another aspect, the present invention provides fuel compositions for internal combustion engines that include a hydrocarbon fuel and a fuel additive comprising amides, wherein the additive and/or fuel is ester free or the molar ratio of amides to esters is greater than 1.4 to 1.

The additive composition of this invention can be prepared by mixing the same (a) with the fuel detergent (b) and, optionally, liquid carrier (c) either sequentially or in any suitable order. For example, the additive can be combined with the Mannich base and then this mixture is combined with the liquid carrier or a mixture of Mannich base and liquid carrier can be combined with the additive. This mixing can take place before the addition of the composition to the fuel or during the mixing of a fuel containing the additive composition of this invention. The order of addition and/or combinations of the various components of this invention is therefore not critical and all such orders of addition and/or combination of the components are envisioned as being within the scope of the invention herein.

In the fuel additive composition and/or fuel composition of this invention, other fuel additives can be employed to enhance the performance of the fuel, including, for example, antioxidants, corrosion inhibitors, dehazers, demulsifiers, metal deactivators, antifoaming agents, combustion improvers such as cetane improvers, co-solvents, package compatibilisers, metallic-based additives such as metallic combustion improvers, anti-knock agents such as tetraethyl lead compounds, anti-icing additives, dyes, one or more fuel-soluble antioxidants, octane improvers, emission reducers, ancillary detergent/dispersant additives, and the like and mixtures thereof.

The fuel additive composition of this invention is particularly useful when employed as an additive in an internal combustion engine fuel composition to improve the delivery of a friction modifier to the combustion chamber and crankcase lubricant. Thus, the fuel composition will contain a major amount of an internal combustion engine fuel and a minor effective amount of at least one fuel additive composition of this invention. In general, the amount of the fuel additive composition employed in the fuel composition can range from about 20 ppm to about 2000 ppm, more preferably 50-1000 ppm and most preferably 500 ppm.

The fuel in which the additive composition of the invention can be used can be any hydrocarbon fuel, e.g., diesel, gasoline, kerosene, jet fuels, etc.; alcoholic fuels such as methanol or ethanol; or, a mixture of hydrocarbon and alcoholic fuels. When the fuel is diesel, such fuel generally boils above about 212° F. The diesel fuel can comprise atmospheric distillate or vacuum distillate, or a blend in any proportion of straight run and thermally and/or catalytically cracked distillates. Preferred diesel fuels have a cetane number of at least 40, preferably above 45 and more preferably above 50. The diesel fuel can have such cetane numbers prior to the addition of any cetane improver. The cetane number of the fuel can be raised by the addition of a cetane improver.

When the fuel is gasoline, it can be derived from straight-chain naphtha, polymer gasoline, natural gasoline, catalytically cracked or thermally cracked hydrocarbons, catalytically reformed stocks, and the like. It will be understood by one skilled in the art that gasoline fuels typically boil in the range of from about 80° F. to about 450° F. and can consist of straight chain or branched chain paraffins, cycloparaffins, olefins, and aromatic hydrocarbons and any mixture of these.

Generally, the composition and octane or cetane level of the fuels are not critical and any conventional fuel can be employed herein. A fuel composition containing the fuel additive composition of the invention is suitable for the operation of an internal combustion engine. When the base fuel is diesel, the fuel composition will be suitable for use in, e.g., compression-ignition engines typically operated on such fuels. When the base fuel is gasoline, the fuel composition will be suitable for use in, e.g., spark-ignition engines typically operated on such fuels. It is to be understood that the fuel compositions containing the fuel additive composition of this invention can be used to operate a variety of engines and in any other application requiring a fuel having improved delivery of friction modifier, e.g., jet engines, furnaces, etc.

The above disclosure describes several preferred embodiments of the invention. The skilled artisan will recognize that other embodiments of this invention, which are not overtly disclosed, may be employed in the practice of this invention. The invention is further illustrated by the following non-limiting examples, wherein all parts are parts by weight unless otherwise specified.

### TEST EXAMPLES

The following test experiments evaluate fuel additives having alkanolamides therein with varying amide to ester molar ratios. The additives having high amide to ester ratios yielded unexpected improvements in fuel economy when tested in vehicles.

Additives Tested

| | Percent | | Amide-to-Ester |
|---|---|---|---|
| Additive | Amide | Percent Ester | Ratio |
| A | 61% | 39% | 1.56:1 |
| B | 99% | 1% | 99:1 |
| C | 58% | 42% | 1.4:1 |
| D | 80% | 20% | 4:1 |
| E | 90% | 10% | 9:1 |

Test protocols

### HWFET Test

The HWFET test is a chassis dynamometer test using the EPA Highway Fuel Economy Test Protocol. This test schedule represents a mixture of rural and interstate highway driving with a warmed-up engine, typical of longer trips in free-flowing traffic. Average test speed is approximately 48 mph and does not include stops or idling, thus minimizing driver error. Triplicate repeats of the 10.25 mile test are conducted using base fuel and then additized fuel in the same vehicle. Fuel economy is calculated per EPA protocol.

### On-Road 56 Mile

The additives were evaluated for performance as fuel economy enhancers in on-road vehicle tests. A test course of 56.5 miles was chosen which had generally light traffic conditions, lack of stops that can increase driver variability, and multiple lanes reducing the likelihood of test vehicle speed being impacted by other traffic. The vehicles fuel pump was turned off by removing the fuel pump fuse. All accessory electronics such as radio, air conditioner, and lights were turned off. The fuel line from the vehicle fuel tank to the engine was sealed and a new fuel line was run from the test equipment to the engine. Fuel was provided to the vehicle from a 5 gallon can with quick disconnect fittings. The fuel rail pressure was matched by a fuel pump in the test equipment. In order to minimize vehicle speed variation, the driver employed the vehicle cruse control at 55 mph. Overall test time and various segments were measured to check vehicle speed.

The fuel container was weighed before and after the test course to determine fuel usage. Following a warmup of greater than 50 miles, the fuel economy of the vehicle was determined for base fuel. The base fuel was fuel not containing a friction modifying fuel additive. The fuel was then switched to base fuel with the test additive and, again following a warmup/conditioning run of greater than 50 miles on the test fuel, the fuel economy of the vehicle operating on fuel containing the test additive was determined.

### On-Road 30 Mile

To evaluate the relative performance of multiple additives, a 30 mile version of the on-road 56 mile test was developed. The 30 mile test was designed so that the vehicle is not turned off between runs, but was otherwise similar to the 56 mile on road test.

### Data

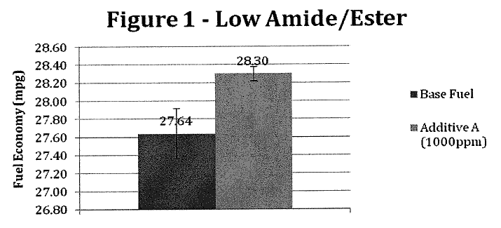

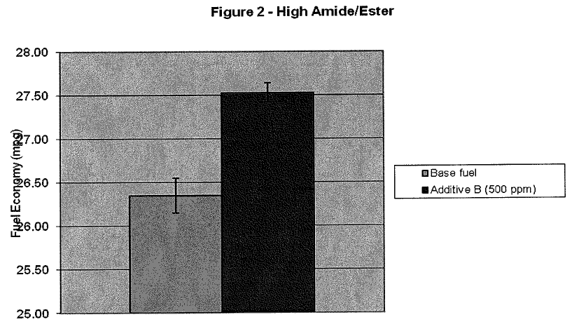

FIG. 1 shows triplicate HWFET results for Additive A (low Amide/ester) evaluated at a 1000 ppm concentration in the test fuel. FIG. 2 shows triplicate HWFET results for Additive B (high Amide/ester) evaluated at a 500 ppm concentration in the test fuel. The 95% confidence interval is shown on each data bar demonstrating statistical significance. Table 1 shows the fuel economy benefits of Additives A and B evaluated in 2008 model year Dodge Caravans equipped with a 3.8 L V6 engine.

TABLE I
fuel economy data

| | | Fuel Economy | |
|---|---|---|---|
| Additive | Dose (ppm) | Benefit (%) | Test |
| A | 500 | 2.4% | On-Road 56 |
| | | | mile |
| A | 1000 | 2.4% | HWFET |
| B | 500 | 4.5% | HWFET |

As can be seen from the above tests, the high amide-to-ester ratio additive (additive B) performed unexpectedly better as a fuel economy additive. However, when the dose rate of Additive A was doubled, thus doubling the amount of amide and ester in the fuel, the friction modification did not improve. Thus, unexpectedly, the higher ratio of amide-to-ester in the additive increases performance, not merely the increase in the amount of amide.

The relative performance of different amide-to-ester ratio additives were evaluated in a 2008 Dodge Caravan equipped with the 3.3 L V6 engine in the 30 mile on-road test. In this test Additive D (4:1 Amide-to-Ester) was compared to Additive C (1.4:1 Amide-to-Ester) and the Additive E (9:1 Amide-to-Ester) was compared to Additive D (4:1 Amide-to-Ester). Table II shows the results. Each of the additives were evaluated at a 500 ppm dose rate in the same base fuel.

TABLE II
Relative additive performance

| Base | Comparison Additive (A-E | Percent fuel economy |
|---|---|---|
| Additive (A-E ratio) | ratio) | improvement (%) |
| Additive C (1.4:1) | Additive D (4:1) | 1.6 |
| Additive D (4:1) | Additive E (9:1) | 2.0 |

The data in Table II demonstrates the continuous improvement in performance with increasing amide-to-ester ratio. Thus, the higher the ratio of amide to ester, the better the fuel economy and friction modification. The absence of esters will achieve optimum results.

It should be understood that the embodiments set forth herein are illustrative and that various modifications may be made to the disclosure herein without departing from the invention. For example, the reaction conditions, treat levels, detergent levels and other variations set forth herein may be modified without departing from the scope of the invention.

What is claimed is:

1. A fuel additive for providing friction modification in an internal combustion engine comprising a friction modifying amount of alkanolamides, wherein the additive is free of esters or the molar ratio of amides to esters within said additive is greater than 1.4 to 1.

2. The fuel additive of claim 1 wherein the molar ratio of amides to esters is greater than 2 to 1.

3. The fuel additive of claim 2 wherein the molar ratio of amides to esters is greater than 4 to 1.

4. The fuel additive of claim 3 wherein the molar ratio of amides to esters is between 9 to 1 and 20 to 1.

5. The fuel additive of claim 1 wherein the molar ratio of amides to esters is between 20 to 1 and 50 to 1.

6. The fuel additive of claim 1 wherein the molar ratio of amides to esters is greater than 50 to 1.

7. The fuel additive of claim 1 wherein said additive comprises a reaction product of the reaction of one or more of a fatty acid, oil and fatty acid ester, with an alkanolamine.

8. A fuel additive according to claim 7, wherein the alkanolamine is a primary and/or secondary amine selected from the group consisting of ethanolamine, propanolamine, isopropanolamine, butanolamine, isobutanolamine, methylethanolamine, dimethylethanolamine, butylethanolamine, diethanolamine, dipropanolamine, diisopropanolamine, dibutanolamine, diisobutanolamine, and mixtures thereof.

9. A fuel additive according to claim 8 wherein the alkanolamine is diethanolamine.

10. A fuel additive according to claim 8, wherein the one or more acid, oil and fatty acid ester comprises a C₆-C₂₂ carboxylic acid obtained from the group consisting of canola oil, castor oil, cocoa butter, coconut oil, cotton seed oil, olive oil, palm kernel oil, palm kernel (olein), palm kernel (stearine), peanut oil, rapeseed oil, safflower oil, soybean oil, sunflower oil, tall oil, corn oil, butter, lard, tallow, yellow grease, blubber, herring, menhaden, sardine oil, babasso oil and mixtures thereof.

11. A fuel additive according to claim 10, wherein the carboxylic acid is a C₁₂-C₁₈ carboxylic acid selected from the group consisting of lauric, cis-9-dodecenoic, myristic, myristoleic, cis-9-tetradecenoic, pentadecanoic, palmitic, palmitoleic, cis-9-hexadecenoic, heptadecanoic, heptadecenoic, steric, oleic, linoleic, linolenic, ricinoleic, dihydroxystearic, and mixtures thereof.

12. The fuel additive of claim 3 wherein said additive comprises a reaction product of the reaction of one or more of a fatty acid, oil and fatty acid ester, with an alkanolamine.

13. A fuel additive according to claim 12, wherein the alkanolamine is a primary and/or secondary amine selected from the group consisting of ethanolamine, propanolamine, isopropanolamine, butanolamine, isobutanolamine, methylethanolamine, dimethylethanolamine, butylethanolamine, diethanolamine, dipropanolamine, diisopropanolamine, dibutanolamine, diisobutanolamine, and mixtures thereof.

14. A fuel additive according to claim 13 wherein the alkanolamine is diethanolamine.

15. A fuel additive according to claim 13, wherein the one or more acid, oil and fatty acid ester comprises a C₆-C₂₂ carboxylic acid obtained from the group consisting of canola oil, castor oil, cocoa butter, coconut oil, cotton seed oil, olive oil, palm kernel oil, palm kernel (olein), palm kernel (stearine), peanut oil, rapeseed oil, safflower oil, soybean oil, sunflower oil, tall oil, corn oil, butter, lard, tallow, yellow grease, blubber, herring, menhaden, sardine oil, babasso oil and mixtures thereof.

16. A fuel additive according to claim 15, wherein the carboxylic acid is a C₁₂-C₁₈ carboxylic acid selected from the group consisting of lauric, cis-9-dodecenoic, myristic, myristoleic, cis-9-tetradecenoic, pentadecanoic, palmitic, palmitoleic, cis-9-hexadecenoic, heptadecanoic, heptadecenoic, steric, oleic, linoleic, linolenic, ricinoleic, dihydroxystearic, and mixtures thereof.

17. The fuel additive of claim 6 wherein said additive comprises a reaction product of the reaction of one or more of a fatty acid, oil and fatty acid ester, with an alkanolamine.

18. A fuel additive according to claim 17, wherein the alkanolamine is a primary and/or secondary amine selected from the group consisting of ethanolamine, propanolamine, isopropanolamine, butanolamine, isobutanolamine, methylethanolamine, dimethylethanolamine, butylethanolamine, diethanolamine, dipropanolamine, diisopropanolamine, dibutanolamine, diisobutanolamine, and mixtures thereof.

19. A fuel additive according to claim 18 wherein the alkanolamine is diethanolamine.

20. A fuel additive according to claim 18, wherein the one or more acid, oil and fatty acid ester comprises a C₆-C₂₂ carboxylic acid obtained from the group consisting of canola oil, castor oil, cocoa butter, coconut oil, cotton seed oil, olive oil, palm kernel oil, palm kernel (olein), palm kernel (stearine), peanut oil, rapeseed oil, safflower oil, soybean oil, sunflower oil, tall oil, corn oil, butter, lard, tallow, yellow grease, blubber, herring, menhaden, sardine oil, babasso oil and mixtures thereof.

21. A fuel additive according to claim 20, wherein the carboxylic acid is a C₁₂-C₁₈ carboxylic acid selected from the group consisting of lauric, cis-9-dodecenoic, myristic, myristoleic, cis-9-tetradecenoic, pentadecanoic, palmitic, palmitoleic, cis-9-hexadecenoic, heptadecanoic, heptadecenoic, steric, oleic, linoleic, linolenic, ricinoleic, dihydroxystearic, and mixtures thereof.

22. The fuel additive of claim 1 wherein said additive comprises

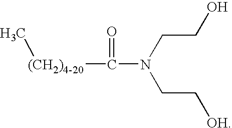

23. The fuel additive of claim 3 wherein said additive comprises

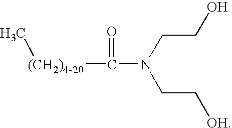

24. The fuel additive of claim 7 wherein said additive comprises

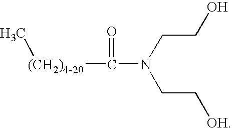

25. A fuel additive according to claim 1 further comprising a fuel detergent chosen from one or more of Mannich base detergents, polyetheramines, polyolefin-amines, poly-olefin polyamines, poly-olefin-phenolpolyamines, and polyolefin succinimides.

26. A fuel additive according to claim 2 further comprising a fuel detergent chosen from one or more of Mannich base detergents, polyetheramines, polyolefin-amines, poly-olefin polyamines, poly-olefin-phenolpolyamines, and polyolefin succinimides.

27. A fuel additive according to claim 3 further comprising a fuel detergent chosen from one or more of Mannich base detergents, polyetheramines, polyolefin-amines, poly-olefin polyamines, poly-olefin-phenolpolyamines, and polyolefin succinimides.

28. A fuel composition comprising
a) a major amount of an internal combustion engine hydrocarbon fuel; and
b) a minor amount of a fuel additive comprising a friction modifying amount of alkanolamides, wherein the additive is free of esters or the molar ratio of amides to esters within said additive is greater than 1.4 to 1.

29. The fuel composition of claim 28 wherein the additive is free of esters or the molar ratio of amides to esters within said additive is greater than 2 to 1.

30. The fuel composition of claim 29 wherein the additive is free of esters or the molar ratio of amides to esters within said additive is greater than 4 to 1.

31. The fuel composition of claim 30 wherein the additive is between 9 to 1 and 20to 1.

32. The fuel composition of claim 30 wherein the additive is free of esters or the molar ratio of amides to esters is between 20 to 1 and 50 to 1.

33. The fuel composition of claim 30 wherein the additive is free of esters or the molar ratio of amides to esters is greater than 50 to 1.

34. The fuel composition of claim 28 wherein the amount of said fuel additive is from about 20 to 2000 ppm by weight based on the weight of hydrocarbon fuel.

35. The fuel composition of claim 30 wherein the amount of said fuel additive is from about 20 to 2000 ppm by weight based on the weight of hydrocarbon fuel.

36. A method of making a fuel additive comprising:
a) reacting a fatty acid, ester or oil with an alkanolamine, thereby producing one or more reaction products useful as a fuel additive, wherein the additive is free of esters or the ratio of amides to esters within said reaction product is greater than 1.4 to 1.

37. The method of claim 36 wherein the additive is free of esters or the ratio of amides to esters within said reaction product is greater than 2 to 1.

38. The method of claim 37 wherein the additive is free of esters or the ratio f amides to esters within said reaction product is greater than 4 to 1.

39. The method of claim 38 wherein the additive is free of esters or the ratio of amides to esters within said reaction product is between 9 to 1 and 50 to 1.

40. The method of claim 38 wherein the additive is free of esters or the ratio of amides to esters within said reaction product is greater than 50 to 1.

41. The method of claim 36, wherein the fatty acid, ester or oil comprises C₄-C₂₂ carboxylic acid obtained from the group consisting of canola oil, castor oil, cocoa butter, coconut oil, cotton seed oil, olive oil, palm kernel oil, palm kernel (olein), palm kernel (stearine), peanut oil, rapeseed oil, safflower oil, soybean oil, sunflower oil, tall oil, corn oil, butter, lard, tallow, yellow grease, blubber, herring, menhaden, sardine oil, babasso oil and mixtures thereof.

42. The method of claim 41 wherein the carboxylic acid is a C₁₂-C₁₈ carboxylic acid selected from the group consisting of lauric, cis-9-dodecenoic, myristic, myristoleic, cis-9-tetradecenoic, pentadecanoic, palmitic, palmitoleic, cis-9-hexadecenoic, heptadecanoic, heptadecenoic, steric, oleic, linoleic, linolenic, ricinoleic, dihydroxystearic, and mixtures thereof.

43. The method of claim 42, wherein the alkanolamine is chosen from ethanolamine, diethanolamine, dimethylethanolamine, N-methylethanolamine propanolamine, isopropanolamine, dipropanolamine, di-isopropanolamine, butanolamines, aminoethylaminoethanol, and mixtures thereof.

44. The method of claim 43, wherein the alkanolamine is diethanolamine.

45. The method of claim 36, wherein the fuel additive comprises

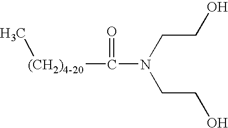

46. The method of claim 38, wherein the fuel additive comprises

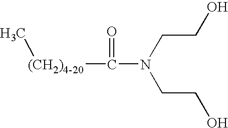

47. The method of claim 40, wherein the fuel additive comprises

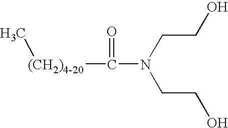

48. The method of claim 36 further comprising:
b) admixing a fuel detergent to the one or more reaction product produced in step (a), wherein the detergent is chosen from one or more of Mannich base detergents, polyetheramines, polyolefin-amines, poly-olefin polyamines, poly-olefin-phenolpolyamines, and polyolefin succinimides.

49. The method of claim 36 further comprising:
b) admixing an internal combustion engine hydrocarbon fuel to the one or more reaction product produced in step (a), wherein the fuel is chosen from gasoline, diesel fuel, kerosene, and jet fuel.

50. The method of claim 38 further comprising:
b) admixing a fuel detergent to the one or more reaction product produced in step (a), wherein the detergent is chosen from one or more of Mannich base detergents, polyetheramines, polyolefin-amines, poly-olefin polyamines, poly-olefin-phenolpolyamines, and polyolefin succinimides.

51. The method of claim 38 further comprising:
b) admixing an internal combustion engine hydrocarbon fuel to the one or more reaction product produced in step (a), wherein the fuel is chosen from gasoline, diesel fuel, kerosene, and jet fuel.

52. The method of claim 40 further comprising:
b) admixing a fuel detergent to the one or more reaction product produced in step (a), wherein the detergent is chosen from one or more of Mannich base detergents, polyetheramines, polyolefin-amines, poly-olefin polyamines, poly-olefin-phenolpolyamines, and polyolefin succinimides.

53. The method of claim 40 further comprising:
b) admixing an internal combustion engine hydrocarbon fuel to the one or more reaction product produced in step (a), wherein the fuel is chosen from gasoline, diesel fuel, kerosene, and jet fuel.

54. A method for enhancing fuel economy of an internal combustion engine, the method comprising
a) fueling the engine with a suitable internal combustion engine hydrocarbon fuel and a fuel additive according to any of claim 1; and
b) operating the engine under normal operating conditions.

55. A method for enhancing fuel economy of an internal combustion engine, the method comprising
a) fueling the engine with a suitable internal combustion engine hydrocarbon fuel and a fuel additive according to any of claim 4; and
b) operating the engine under normal operating conditions.

56. A method for enhancing fuel economy of an internal combustion engine, the method comprising
a) fueling the engine with a suitable internal combustion engine hydrocarbon fuel and a fuel additive according to any of claim 6; and
b) operating the engine under normal operating conditions.

57. The method according to claim 49, wherein the relative proportion of fuel additive to hydrocarbon fuel is from about 0.002 percent by volume to about 0.05 percent by volume.

58. The method according to claim 51, wherein the relative proportion of fuel additive to hydrocarbon fuel is from about 0.002 percent by volume to about 0.05 percent by volume.

59. The method according to claim 53, wherein the relative proportion of fuel additive to hydrocarbon fuel is from about 0.002 percent by volume to about 0.05 percent by volume.
